(19) United States
(12) Patent Application Publication
(10) Pub. No.: US 2016/0045352 A1
(43) Pub. Date: Feb. 18, 2016

(54) GUPPY ERGONOMIC KNEE ASSISTIVE DEVICE

(71) Applicant: Denise Bowen, Palm Desert, CA (US)

(72) Inventor: Denise Bowen, Palm Desert, CA (US)

(21) Appl. No.: 14/827,148

(22) Filed: Aug. 14, 2015

Related U.S. Application Data
(60) Provisional application No. 62/037,530, filed on Aug. 14, 2014.

(51) Int. Cl. A61F 5/01

(52) U.S. Cl. CPC A61F 5/0106

(57) ABSTRACT

A knee assistive device for cushioning and protecting a user is provided. The device comprises a concave base having an inner surface, an outer surface, a front end, a first side, a second side, and a rear end. A side wall extends upward from the base. A knee well is formed in the inner surface of the base adjacent the front end. A foot well is formed in the inner surface of the base adjacent the knee well of the base with the foot well being separate and distinct from the knee well and extending to a point below the knee well. A cushioned pad is positioned within the knee well. Upon positioning of the knees within the knee well and the feet within the foot well, the user rocks back and forth on the base while the knees, calves, ankles, and feet of the user are cushioned and protected.

### CLAIM OF PRIORITY

This patent application claims priority under 35 USC 119 (e) (1) from U.S. Provisional Patent Application Ser. No. 62/037,530 filed Aug. 14, 2014, of common inventorship herewith entitled, “Guppy Ergonomic Knee Assistive Device,” which is incorporated herein by reference as though the same were set forth in its entirety.

### FIELD OF THE INVENTION

The present invention pertains to the field of body protective aids, ergonomic work aids and more specifically to the field of padded knee protectors and assistive devices.

### BACKGROUND OF THE INVENTION

Professional contractors and amateur do-it-yourselfers embark on a variety of construction and home improvement tasks every day, such as building an addition on a home, replacing flooring, repairing plumbing, gardening, painting and maintenance. The results achieved by successfully completing these tasks are enjoyed for years. When completing various construction and home and garden tasks, many workers find that a majority of the time executing these endeavors is spent in a kneeling position. Especially when performing gardening tasks, one positions their body low to the ground to reach plant and garden beds. Kneeling in this manner causes extreme discomfort, even for those in the most optimal physical shape. Resting one's body weight on their knees, coupled with the pressure applied by hard ground surfaces, results in an individual enduring extreme discomfort while completing various tasks. While kneeling for an extended period causes discomfort for able-bodied individuals, this discomfort is exacerbated for senior citizens and persons who suffer medical conditions like arthritis or muscle and joint pain. Being unable to complete household and garden tasks such as planting spring flowers or painting trim around residential floor boards because of stiff muscles and extreme sore joints detracts from the joy and accomplishment associated with these endeavors.

The prior art has put forth several designs for padded knee protectors. Among these are:

U.S. Pat. No. 3,499,502 to Judge E. Rosander describes a kneeling device for use by cement finishers and others comprising a smooth flat base having upwardly and inwardly curved end portions which constitute handles extending across the entire width of the base. A kneeling pad on the base is spaced from the ends of the base to provide spaces which make the inner surfaces of the handles accessible for engagement by the fingers of a user or a tool for lifting or moving the device. The pad is made of a non absorbent, non porous, resilient material which is instantly expansible when pressure applied thereto has been released and which yieldingly and comfortably supports the knees of the user without bottoming. This kneeling device is moved easily from one place to another, is slidable over a supporting surface, is impervious to moisture and provides comfortable support for the knees of the user. The base of the kneeling device preferably is made of smooth plastic material which is moldable to provide continuous handle means at opposite ends. These handle grooves are sufficiently spaced from the kneeling pad to provide a user easy access to the handles in one of two ways, by grasping with their hands or by engaging a trowel or tool to lift or move the kneeling device as a whole. For most convenient use in cement finishing or similar tasks, two of the devices are employed, the first one for the worker to kneel upon and the second one for his or her feet to rest upon during one stage of the work. When that stage is completed, the worker moves backward to kneel on the second device and then moves the first one rearwardly to support his feet and prevent contact with the cement being finished.

U.S. Pat. No. 2,124,158 to Douglas Fredrick Turner describes a knee pad comprising a semi rigid flexible casing substantially L shaped and horseshoe shaped in side and plan views. The horizontally elongated portion constitutes the main pad and the upstanding vertical frontal portion constitutes a knee abutment. The said abutment and companion horizontal portion are channel shaped in cross sectional form. The top wall of said pad has a slit therein forming an aperture for insertion and removal of a pneumatic bladder. The pneumatic bladder in said casing is proportionately and substantially commensurate with the surrounding casing to constitute fillers for the main pad as well as said abutment. The said bladder has at one end an inflation valve with the said valve being exposed through the crown portion of said abutment.

U.S. Pat. No. 2,448,427 to Benjamin Gordon describes a knee pad dolly which is comprised of a platform or frame supported by four ball bearing casters affixed to each corner of the underneath side of the dolly. The casters are thus configured so they are free to move in any direction, enabling the operator to move and change the position of the dolly with a minimum of effort. The platform, which is roughly rectangular in shape, is stamped from one sheet of metal resulting in the formation of the four sides that extend for a short distance from the base of the platform. Around the top portion of the four sides is a rolled edge which is either formed from the sides or by welding a lightweight metal tube to the top edge of the sides. This results in the formation of a dolly that is attractive in appearance and contains rounded edges to protect a user and also prevents furniture or other similar objects from being marred by contact with a sharp edge.

None of these prior art references describe the present invention.

### SUMMARY OF THE INVENTION

It is an object of the present invention to provide both an ergonomic and padded platform on which to kneel when working low to the ground and as an assistive device to help the user get up from and down into a kneeling attitude.

The present invention is a knee assistive device for cushioning and protecting knees, calves, ankles, and feet of a user. The knee assistive device comprises a substantially concave base having an inner surface and an outer surface with the base further having a front end, a first side, a second side, and a rear end. A side wall extends upward from the base. A knee well is formed in the inner surface of the base adjacent the front end of the base. A foot well is formed in the inner surface of the base adjacent the knee well of the base with the foot well being separate and distinct from the knee well and extending to a point below the knee well. A cushioned pad is positioned within the knee well. Upon positioning of the knees within the knee well and the feet within the foot well, the user is able to rock back and forth on the concave base while the knees, calves, ankles, and feet of the user are cushioned and protected.

The present invention further includes a knee assistive device for cushioning and protecting knees, calves, ankles, and feet of a user. The knee assistive device comprises a substantially concave base having an inner surface and a smooth, slick outer surface with the base further having a front end, a first side, a second side, and a rear end. A side wall extends upward from the base with the side wall having a top edge with has an integrated, rounded hand rail. The hand rail has a thickness greater than a thickness of the side wall. A knee well is formed in the inner surface of the base adjacent the front end of the base. A foot well is formed in the inner surface of the base adjacent the knee well of the base with the foot well being separate and distinct from the knee well and extending to a point below the knee well. A cushioned pad is positioned within the knee well and a storage compartment is formed on an inside surface of the side wall. The inner surface dips gently downward from the knee well transitioning into the integrated foot well. Upon positioning of the knees within the knee well and the feet within the foot well, the user is able to rock back and forth on the concave base while the knees, calves, ankles, and feet of the user are cushioned and protected.

### BRIEF DESCRIPTION OF THE DRAWINGS

FIG. 1 is an illustrative rear perspective view showing the interior gel cushion and interior elements of the present invention.

FIG. 2 is a diagrammatic side view showing interior structure of the present invention including curved interior lip to hold while carrying over the shoulder, interior floor, foot well, negative space arch under entry ramp, integrated carrying handle and/or “rod” for hanging on the wall, entrance ramp, and inner separation of tool/toy storage.

FIG. 3 is a diagrammatic side view of the present invention.

FIG. 4 is an illustrative side view showing the hand rail, shaped hand grip, handle, storage compartment and safety stop of the present invention.

FIG. 5 is a perspective view illustrating the hand rail of the present invention.

FIG. 6 is a perspective view illustrating the hand grip of the present invention.

FIG. 7 is a perspective view illustrating the handle of the present invention.

### DETAILED DESCRIPTION OF THE INVENTION

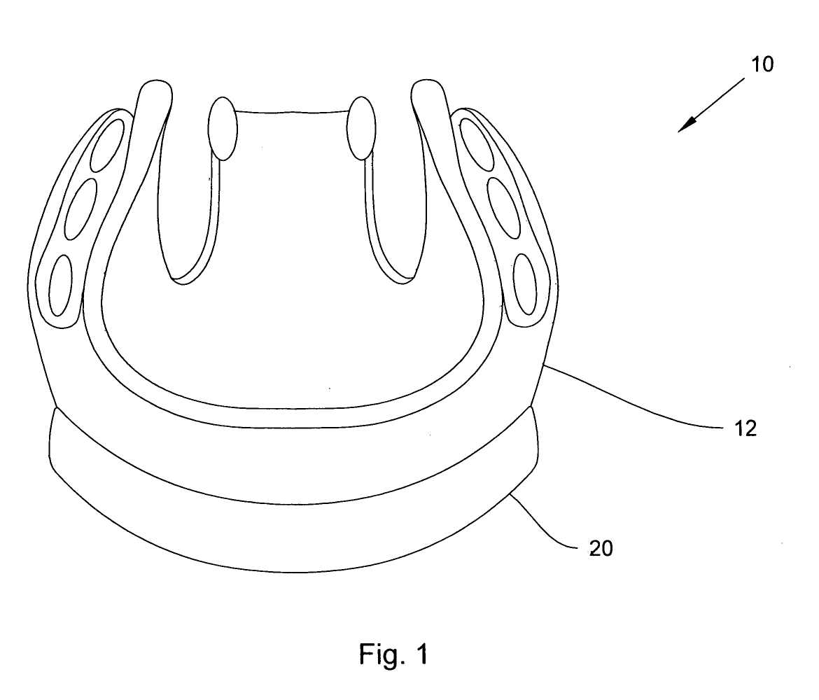

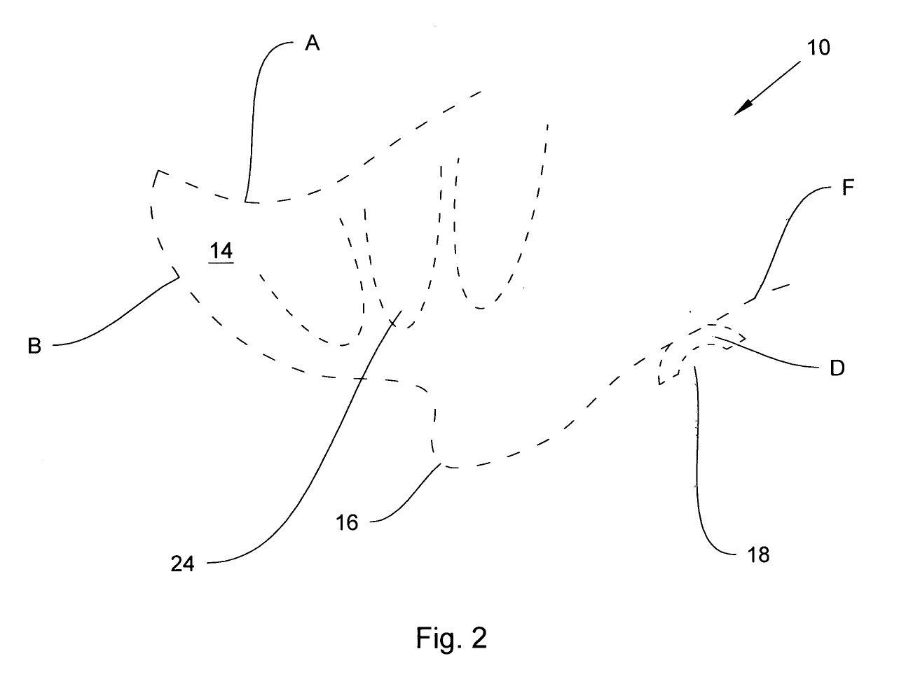

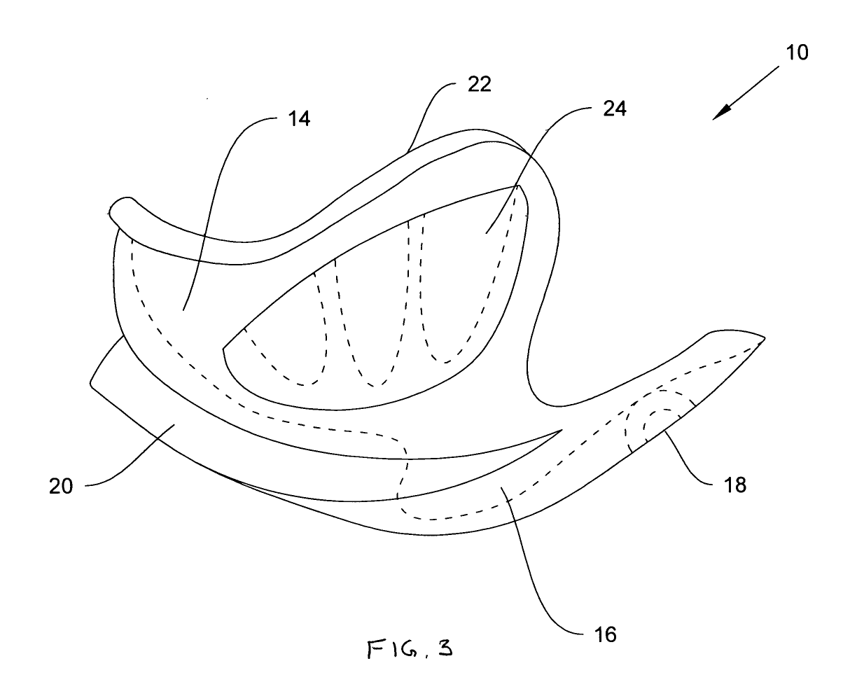

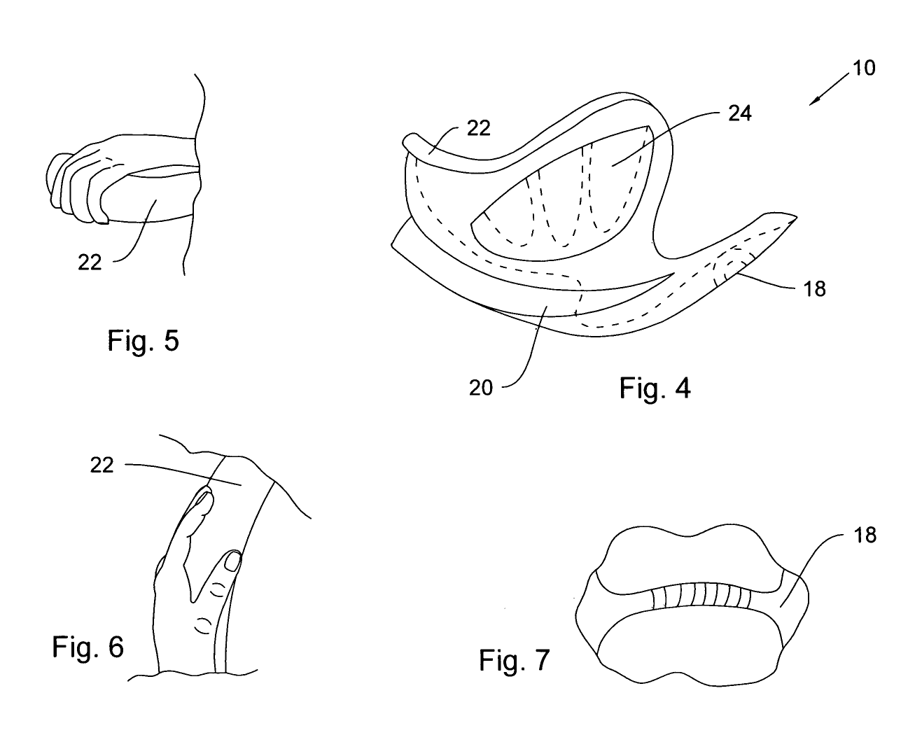

As illustrated in FIGS. 1-7, the present invention, hereinafter referred to as the Guppy Ergonomic Knee Assistive Device, indicated generally at 10, is an ergonomic, assistive padded platform providing users with cushiony comfort to their knees, shins and ankles while allowing the feet flexibility and freedom of movement, when gardening, kneeling or working low to the ground. The knee assistive device 10 can be designed and constructed to receive a single leg or both legs of the user.

The knee assistive device 10 utilizes both ergonomic grips and motion to help the user get up and down while working or completing home improvement or maintenance tasks. The knee assistive device 10 provides reliable comfort and support to a person's lower extremities and knees when kneeling and also cushioning and supporting one's knees, shins and ankles from hard surface areas during use. The knee assistive device 10 is produced in two to three sizes to accommodate various users including adults and children. The knee assistive device 10 of the present invention is an elevated platform comprising a rounded base 12 that enables the present invention to slightly rock during use. The knee assistive device 10 contains an ample, padded support area 14 for the knees. Behind the padded support area 14 is a recessed foot well 16 that accommodates the user's feet. Preferably, a front edge of the foot well 16 is positioned approximate geometric center of the concave base 12 promoting better balance of the device 10. Oval in shape, the front end of the knee assistive device 10 tapers inwards towards the rear of the unit and culminates to an integrated handle 18 under the rear ramp.

The standard-sized knee assistive device 10 of the present invention measures approximately thirty nine inches in total length. The kneeler portion of the standard sized device 10 comprises approximately twenty four inches of the total thirty nine inches in length and fourteen inches in depth. The foot well portion of the standard sized device 10 comprises approximately fifteen inches of the total thirty nine inches in length and fifteen inches in depth.

The kneeling area 14 of the knee assistive device 10 of the present invention contains a cushiony soft and supportive padding comprised of dense memory foam or gel material measuring a minimum of approximately three inches in depth. The long-lasting cushions are replaceable and have durable, protective and water-resistant covers which can be easily removed for cleaning. The interior base of the device 10 is a soft plastic shell curved to create a unique, body-assistive W shape, while the distal end of the kneeler portion 14 dips gently downward transitioning into the integrated foot well 16. The exterior base 12 is a hard, curved plastic shell the underside of which has a semi-slick surface that both facilitates mobility and discourages insects from climbing up and into the present invention. A retaining, wedge-like lip 20 integrally encircles base 12 and serves as a brake so the user can never lean too far forward. The bottom of the retaining lip 20 is covered in a non-skid material and ridged for stability during use. These features combine to provide comfort and safety while enabling a user to easily rotate the present invention in any direction as well as gently rock it forward to securely assist with leaning in to complete a specific task.

The user's shins and ankles are not only supported by the cushion but are also comfortably positioned to allow the foot and ankle to flex and bend maintaining a natural position and preventing numbness, fatigue and stress while kneeling. The foot well 16 and entrance ramp behind it are covered in a non-skid material and ridged, inside and out, to provide traction, stability and firm footing during use. The ramp area's ridges are slightly curved toward the center of the ramp and then canted toward the outside of the device 10. These runnels allow drainage to the exterior of the unit from a user's wet or dirty boots and keep the interior clean and dry.

There are two ways to enter the knee assistive device 10 of the present invention: from the side and from the rear. From the side a user steps directly into the foot well 16, over the adjacent side wall area which dips to its lowest point here. This allows the user to easily step in and out. The second method is to approach the device 10 from the rear. The distal ramp walls curve gently upward from the lowest point by the foot well 16 and culminate in a flattened and smoothly rounded “step” area. The user lightly steps on either side and gently rocks the ramp of the device 10 down to ground level. The ramp provides both an easy, transitional entry to the unit 10 and a way to avoid getting water or debris in the foot well 16.

While standing on the ramp, users may lean forward and grasp the smoothly rounded upper edge of the device 10, each side of which is correctly shaped to accommodate the right or left heel of the hand. These depressions are integrated into the apexes of both sides of the unit's walls. The rounded upper lip 22 of the device 10 is curved and shaped to allow the user to gently slide his body down into a kneeling position. Conversely, when wanting to stand and exit the unit, the user just rolls backwards by a simple movement of his feet into a standing position. The upper lip 22, by its smoothness and shape, gives an assist to a standing position.

Application and use of the knee assistive device 10 of the present invention is simple and straightforward. After purchasing a device 10 and bringing it home, the user loads his tools and supplies in a series of integrated, recessed bins 24 on both sides of the device 10 unit where they are easily accessed by the user. These bins 24 have either integrated lids or “rollaway” plastic covers which will keep the contents secure while being transported to the user's work area. There are two ways to carry the device 10: by its integrated handle 18 located beneath the end of the ramp or over the shoulder. Because of its unique design, the user can place the front, inside edge of the invention over his shoulder and the device's 10 curves and contours anatomically match the form of the user. An integrated hand-hold is positioned under and inside the front lip on both sides of the unit to assist with over-the-shoulder carrying. The lightweight device 10 is easily transported to the user's work location. One positions the device 10 on the ground or floor directly in front of their work area. After positioning the device 10, a user may either enter the unit by the side or back and, using the curved, upper lip and a gentle rocking motion, easily lowers himself into a kneeling position on the deep, knee-embracing cushion. The user leans into his work in a natural manner and the device 10 moves with the motion, gently tilting forward or sideways according to the needs of the consumer. The user may use this rocking motion and the slick bottom surface to move to another work area as desired. After the designated task is complete, the user grasps the ergonomically shaped upper lip and, rolling his feet and rocking gently backward, the user easily stands up and steps out of the device 10. The device 10 is utilized when necessary and, when unneeded, stored away with other tools and accessories or hung up and out of the way by the integrated hand-holds under the lip or the handle 18 under the ramp.

An ergonomic and densely padded platform, shaped for the knees and lower body, the knee assistive device 10 of the present invention cushions the knees, shins and ankles and allows the foot to be held in a natural, flexed and unrestrained position during the course of one's endeavors. Whether gardening, painting trim or even installing flooring, the device evenly and naturally distributes the weight and stresses of the lower extremities. The device 10 also provides a reliable barrier between the user and hard surfaces such as flooring and ground, insects and hot, wet or cold surfaces. Durably constructed of quality materials and with replaceable cushions and cushion covers, the device will withstand years of reliable use with ease.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A knee assistive device for cushioning and protecting knees, calves, ankles, and feet of a user, the knee assistive device comprising:
a substantially concave base having an inner surface and an outer surface, the base further having a front end, a first side, a second side, and a rear end;
a side wall extending upward from the base;
a knee well formed in the inner surface of the base adjacent the front end of the base;
a foot well formed in the inner surface of the base adjacent the knee well of the base, the foot well being separate and distinct from the knee well and extending to a point below the knee well; and
a cushioned pad positioned within the knee well;
wherein upon positioning of the knees within the knee well and the feet within the foot well, the user is able to rock back and forth on the concave base while the knees, calves, ankles, and feet of the user are cushioned and protected.

2. The knee assistive device of claim 1 wherein the side wall extends only along a portion of the first side and the second side wall from the front end to the rear end of the base.

3. The knee assistive device of claim 2 wherein the side wall extends to a point past the foot well toward the rear end of the base.

4. The knee assistive device of claim 1 wherein the side wall curves upward from the front end toward the rear end of the base.

5. The knee assistive device of claim 1 wherein a top edge of the side wall has an integrated, rounded hand rail having a thickness greater than a thickness of the side wall.

6. The knee assistive device of claim 1 and further comprising:
a storage compartment formed on an inside surface of the side wall.

7. The knee assistive device of claim 1 wherein the outer surface of the base has a smooth, slick surface.

8. The knee assistive device of claim 1 and further comprising a handle formed in the outer surface of the base adjacent the rear end of the base.

9. The knee assistive device of claim 1 wherein a front edge of the foot well is positioned approximate geometric center of the concave base.

10. The knee assistive device of claim 1 wherein the inner surface dips gently downward from the knee well transitioning into the integrated foot well.

11. The knee assistive device of claim 1 and further comprising:
a retaining, wedge-like lip integrally encircling the base, the lip inhibiting the base from rocking forward past a predetermined point.

12. The knee assistive device of claim 11 wherein the retaining lip is covered in a non-skid material and ridged for stability during use.

13. The knee assistive device of claim 1 the foot well and the inner surface between the foot well and the rear end of the base is covered in a non-skid material and ridged thereby providing traction, stability and firm footing during use.

14. The knee assistive device of claim 1 wherein the inner surface between the foot well and the rear of the base is slightly curved toward the center of the inner surface and canted toward the first side and the second side of the base.

15. A knee assistive device for cushioning and protecting knees, calves, ankles, and feet of a user, the knee assistive device comprising:
a substantially concave base having an inner surface and a smooth, slick outer surface, the base further having a front end, a first side, a second side, and a rear end;
a side wall extending upward from the base, the side wall having a top edge with has an integrated, rounded hand rail, the hand rail having a thickness greater than a thickness of the side wall;
a knee well formed in the inner surface of the base adjacent the front end of the base;
a foot well formed in the inner surface of the base adjacent the knee well of the base, the foot well being separate and distinct from the knee well and extending to a point below the knee well;
a cushioned pad positioned within the knee well;
a storage compartment formed on an inside surface of the side wall;
wherein the inner surface dips gently downward from the knee well transitioning into the integrated foot well; and
wherein upon positioning of the knees within the knee well and the feet within the foot well, the user is able to rock back and forth on the concave base while the knees, calves, ankles, and feet of the user are cushioned and protected.

16. The knee assistive device of claim 15 wherein the side wall extends only along a portion of the first side and the second side wall from the front end to the rear end of the base.

17. The knee assistive device of claim 15 and further comprising a handle formed in the outer surface of the base adjacent the rear end of the base.

18. The knee assistive device of claim 15 wherein a front edge of the foot well is positioned approximate geometric center of the concave base.

19. The knee assistive device of claim 15 and further comprising:
a retaining, wedge-like lip integrally encircling the base, the lip inhibiting the base from rocking forward past a predetermined point;
wherein the retaining lip is covered in a non-skid material and ridged for stability during use.

20. The knee assistive device of claim 15 wherein the inner surface between the foot well and the rear of the base is slightly curved toward the center of the inner surface and canted toward the first side and the second side of the base.
